(19) United States
(12) Patent Application Publication
(10) Pub. No.: US 2003/0113767 A1
(43) Pub. Date: Jun. 19, 2003

(54) Confirmation sequencing

(76) Inventors: Marc J. Rubenfield, Framingham, MA (US); Anuradha Thangavelu Truax, Cambridge, MA (US); Christopher P. Strassel, East Bridgewater, MA (US)

Correspondence Address: CAMPBELL & FLORES LLP, 7th Floor, 4370 La Jolla Village Drive, San Diego, CA, 92122, US

(21) Appl. No.: 10/233,070

(22) Filed: Aug. 29, 2002

Related U.S. Application Data
(60) Provisional application No. 60/316,077, filed on Aug. 29, 2001.

(51) Int. Cl. C12Q 1/68; G06F 19/00; G01N 33/48; G01N 33/50

(52) U.S. Cl. 435/6; 702/20

(57) ABSTRACT

The invention provides an automated method for determining similarity between each of two or more template sequences and a corresponding reference sequence. The method involves the computer implemented steps of (a) verifying that one or more first read sequences corresponding to one or more first portions of each of the two or more template sequences is substantially the same as a corresponding reference sequence, wherein the first read sequences are obtained by sequencing using a defined primer, and (b) confirming that one or more second read sequences corresponding to second portions of one or more verified template sequences is substantially the same as the corresponding reference sequence, wherein the second read sequences are obtained using reference sequence primers.

This application claims benefit of the filing date of U.S. Provisional Application No. 60/316,077, filed Aug. 29, 2001, which is incorporated herein by reference.

### BACKGROUND OF THE INVENTION

There has been a great endeavor among scientists in recent years to determine the sequence of genes in various organisms. Both private and public databases have been developed to assist in this process. To use such data in biological experiments, the gene or sequence of interest is often cloned.

One common method for cloning is polymerase chain reaction (PCR). Although accuracy of PCR products has increased over the past several years due to the discovery of higher fidelity enzymes, errors still occur during the PCR amplification and cloning process. Such errors can result in the generation of a product sequence that differs from the original sequence, and such an incorrect product sequence can encode an incorrect translation product. Therefore, it is advisable for scientists to confirm that a particular clone matches the expected gene.

Additionally, the human genome project has generated databases containing thousands of sequences. However, many of these sequences are riddled with errors. This fact hinders gene expression experiments when scientists produce clones that may differ from the published sequences. Thus, confirming the correctness of these sequences is advisable. Further, microarray technology has allowed for the generation of chips, plates and slides that contain a multitude of cloned gene sequences. To produce high quality microarrays it is advisable to confirm the correctness of each cloned sequence in the microarray.

However, in spite of advances in sequencing technology, a number of problems exist with current methods for confirming the sequence of clones. Specifically, the currently used methods are not fully automated and require human intervention. As a result, when cloning a large number of genes, it is difficult, time consuming, and expensive to confirm that a specific clone has been correctly identified to contain the correct gene. Another problem relates to determining if the cloned sequence has any nucleotide differences from the expected gene sequence. If the cloned sequence does have such discrepancies, the problem of how to determine the significance of those changes arises.

Thus, there is a need for a time- and cost-efficient methods and systems for determining the accuracy of a cloned gene. There is also a need in the art for a method of determining whether discrepancies occur during the cloning process and whether such differences are benign, or lead to changes in amino acid sequence that affect the use of the product of the cloned gene.

### SUMMARY OF THE INVENTION

The invention provides an automated method for determining similarity between each of one or more template sequences and a corresponding reference sequence. The method involves the computer implemented steps of (a) verifying that one or more first read sequences corresponding to one or more first portions of each of the one or more template sequences is substantially the same as a corresponding reference sequence, wherein the first read sequences are obtained by sequencing using a defined primer, and (b) confirming that one or more second read sequences corresponding to second portions of one or more verified template sequences is substantially the same as the corresponding reference sequence, wherein the second read sequences are obtained using reference sequence primers.

In one embodiment, the method also can include an automated step of determining a consensus first or second read sequence. In a further embodiment the method can include identifying one or more differences between one or more template sequence read sequences and the corresponding reference sequences. The identified differences can be between one or more nucleotide sequences and a reference sequence, or between an amino acid sequence encoded by one or more template read sequences or template consensus sequences and an amino acid sequence encoded by the reference sequence.

In another embodiment, the automated method for determining similarity between each of one or more template sequences and a corresponding reference sequence can include assembling a plurality of contiguous read sequences for one or more verified template sequences to generate an assembled template sequence, the read sequences obtained using a plurality of reference sequence primers. The assembled template sequence can be a partial or full length template sequence, and can be single-stranded or double-stranded.

The invention also provides a computer readable medium comprising instructions, which when executed on a processor, implement a method comprising the computer implemented steps: (a) verifying that one or more first read sequences corresponding to one or more first portions of each of the one or more template sequences is substantially the same as a corresponding reference sequence, wherein the first read sequences are obtained by sequencing using a defined primer, and (b) confirming that one or more second read sequences corresponding to second portions of one or more verified template sequences is substantially the same as the corresponding reference sequence, wherein the second read sequences are obtained using reference sequence primers.

The invention further provides an automated system for determining similarity between each of one or more template sequences and a corresponding reference sequence. The system includes (a) a verification module to verify that one or more first read sequences corresponding to one or more first portions of each of the one or more template sequences is substantially the same as a corresponding reference sequence, wherein the first read sequences are obtained by sequencing using a defined primer, and (b) a confirmation module to confirm that one or more second read sequences corresponding to second portions of one or more verified template sequences is substantially the same as the corresponding reference sequence, wherein the second read sequences are obtained using reference sequence primers.

### BRIEF DESCRIPTION OF THE DRAWINGS

FIG. 1 depicts a typical sequencing scheme for determining whether a clone sequence, or an unknown sequence, is substantially the same as a reference sequence and, if not, what nucleotide discrepancies exist. The scheme further depicts a strategy for determining whether those discrepancies lead to an amino acid change upon translation.

FIG. 2 depicts three exemplary scenarios in which a confirmation sequencing process of the invention can be used.

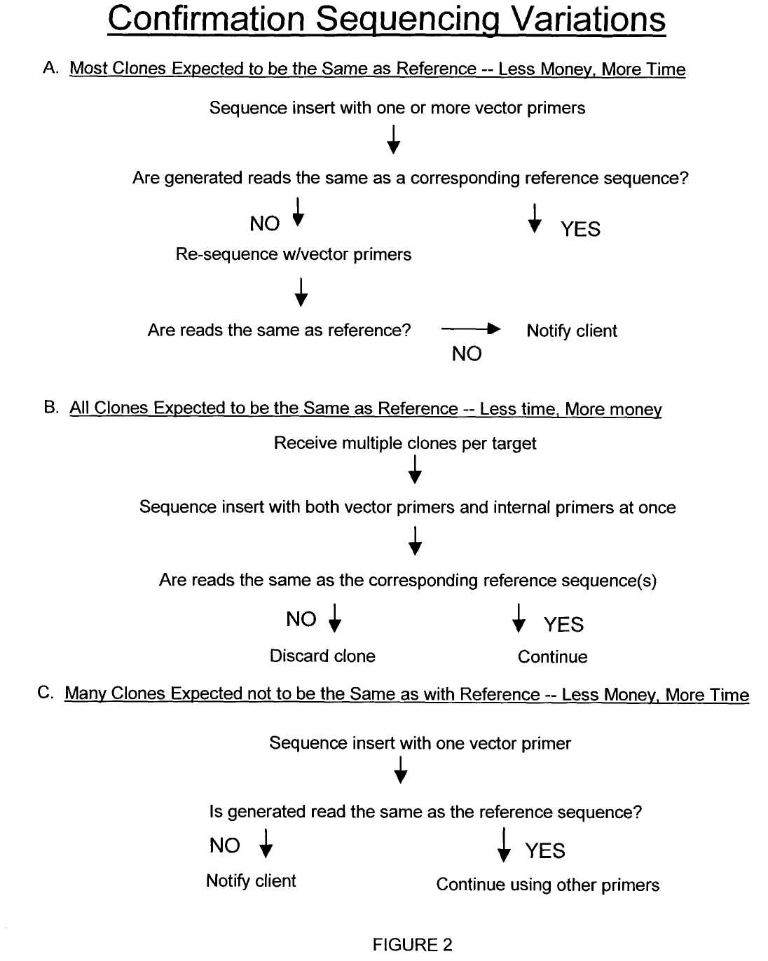

FIG. 3 depicts one practical application of the present invention to confirm correctness of clones expected to encode specific genes.

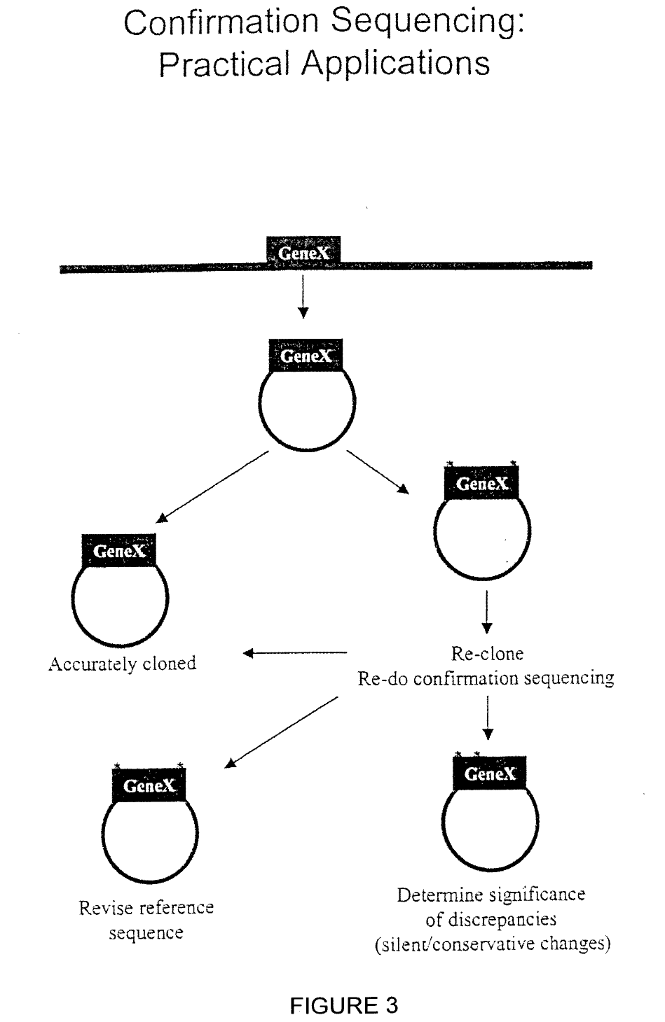

### DETAILED DESCRIPTION OF THE INVENTION

The present invention is directed to an automated method for determining whether a nucleic acid molecule is similar to a defined reference sequence. The method is useful, for example, to determine if a cloned sequence is correct. The method typically involves determining a first read sequence corresponding to a portion of a template sequence, and comparing the obtained sequence with the expected nucleotide sequence, referred to as the corresponding reference sequence. One or more template sequences verified to match with a corresponding reference sequence can then proceed to further sequence analysis. By discarding incorrect template sequences and proceeding only with verified template sequence clones, the methods of the invention save both time and expense.

Additional advantages of the automated method for determining whether a template sequence is the same as a corresponding reference sequence is that the process is fast compared to typical semi-automated sequencing processes, has high capacity, and can involve minimal human intervention, if desired. Therefore, the process can be routinely performed in high throughput formats on a daily or hourly basis.

In one embodiment, the method for determining similarity between one or more template sequences and a corresponding reference sequence can involve a verification step and a confirmation step. In the verification step, one or more primers are used to sequence a portion of each template sequence. It can be convenient to use a vector primer in the verification step because such primers are generally well-characterized and produce consistent results. Each template sequence can be sequenced with a different defined primer or for convenience, with a common defined primer. The resultant first read sequences are then compared with a corresponding reference sequence. Each first read sequence can be compared with a different reference sequence, one or more read sequences can share a common reference sequence, or a combination of individual and shared reference sequences can be used. An automated analysis is performed to determine whether the read sequence has sufficient nucleotide base identity with the corresponding reference sequence to be verified as substantially the same as the reference sequence.

If a first read sequence is determined to be different from the corresponding reference sequence, the template sequence can be considered an incorrect clone and can be discarded without incurring any additional expense to sequence the entire length of the clone. If, on the other hand, the first read sequence is substantially the same as the reference sequence, the clone can be considered to be correct, and sequencing of the clone can proceed to determine whether the clone contains another portion of the reference sequence, or even to determine whether the clone contains the full length of the reference sequence.

When desired to determine if a full length template sequence is similar to a reference sequence, an automated step can be performed in which internal primers from the reference sequence are selected to generate additional or full-length coverage of the reference sequence. The template sequence, which can be a single read sequence or can be assembled from two or more read sequences, can then be sequenced with one or more selected reference sequence primers. The newly generated read sequences are assembled into a larger portion of a template sequence, or a full-length template sequence, and each assembled template sequence can be compared with the corresponding reference sequence.

In another embodiment, the invention is directed to a method for determining the existence of differences in the nucleic acid code of a template sequence in comparison to a reference sequence, and evaluating the effect of those differences on translation of the nucleic acid. In one approach for detecting differences, nucleotide sequences of a template sequence and a reference sequence are compared, as describe above. If the sequences are not substantially the same, one or more differences can be confirmed, for example, by generating high-quality coverage in the opposite direction in the region of the discrepancy. Confirmed differences then can be translated to determine what amino acid change, if any, results. If an amino acid change does occur, the clone can be further analyzed to determine whether the amino acid change is, for example, a conservative change, a silent change, or a non-silent change. This information can then be reported to the client, allowing the client to evaluate the usefulness of the clone for its intended purpose.

In one scenario for using the automated confirmation sequencing method of the invention, most of the clones analyzed are expected to be the same as a corresponding reference sequence. In this situation, the subject sequences can be sequenced using 5′ and 3′ vector primers. If the generated reads are not substantially the same as the corresponding reference sequences, the subject sequences can be re-sequenced using at least one vector primer to confirm that the clone differs from the reference sequence. If the reads from this re-sequencing differ from the reference sequence, the method can be terminated and the client can be notified that the clone does not contain the reference sequence. If the reads of either the original sequencing or re-sequencing with the vector primers are the same as the reference sequence, then confirmation sequencing can be continued using internal reference primers.

In another scenario for using the confirmation sequencing methods of the invention, all of the clones analyzed using the automated methods of the invention are expected to be the same as the reference sequence. In one example, each template sequence is expected to be a clone for a particular target gene. In such a case, the template sequence can be sequenced with both vector primers and internal reference primers at the same time. Because all of the template sequences are expected to be the same as the reference sequence, sequencing with multiple primers is less time consuming than sequencing with each primer individually. If the template reads are not the same as reference sequence, the clone can be discarded. If the reads are the same as the reference sequence, the method can be continued to determine additional read sequences that cover another portion of the template sequences. The process can be continued to determine if any differences between additional template sequence and the reference sequence exist and to determine the effect of any such differences on translation of the clone.

In a further scenario for using the confirmation sequencing methods of the invention, many of the clones analyzed are expected to be different from a corresponding reference sequence. In this situation, the template sequence can be first sequenced with one vector primer. If the generated reads are not the same as the reference sequence, sequencing can be terminated and the client can be notified. If, on the other hand, the reads are substantially the same as the reference sequence, the template sequence can then be sequenced with internal reference primers.

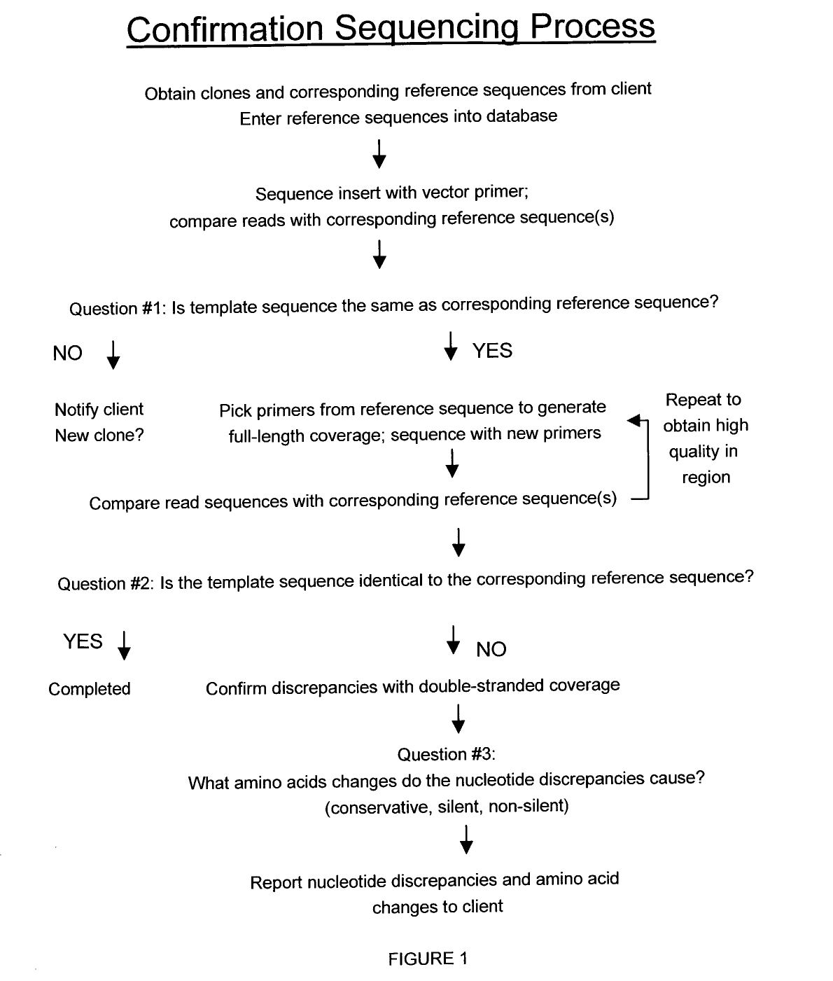

FIG. 1 outlines one exemplary scheme for using a confirmation sequencing method for determining whether a template sequence is similar to a corresponding reference sequence.

As used herein, the term “template sequence” is intended to mean a DNA molecule of interest. A template sequence can include contiguous nucleotide sequences of different types. For example, a template sequence can include vector sequence and clone sequence.

As used herein, the term “reference sequence” is intended to mean to the nucleotide sequence of a defined DNA molecule. A reference sequence can include contiguous nucleotide sequences of different types, such as vector sequence and clone sequence. A reference sequence that corresponds to a given template sequence is a sequence expected to be contained in the particular template sequence, or predicted to be contained in the particular template sequence.

As used herein, the term “assembling” is intended to mean the collecting and fitting together of portions of a nucleic acid sequence into a contiguous sequence representation. A contiguous collection of sequence portions will be represented without redundancy and derived from non-coextensive, overlapping portions of sequence. Therefore, the term is intended to mean a linear, non-redundant electronic representation of a sequence constructed from smaller overlapping sequences or reads corresponding to a nucleic acid molecule.

As used herein, the term “consensus” is intended to mean the reduction of a nucleotide or amino acid position in a multiple alignment to a single inclusive base or residue character. The single inclusive base or residue can represent, for example, a nucleotide or residue occurring at the referenced position that occurs most frequently or is the most likely to occur based on quality scores or error models. Inclusive positions also can include, for example, two or more alternatives at a particular position where the alternatives are equally likely to occur. Consensus sequences can be generated by, for example, by alignment, assembly or other relative comparison of a plurality of nucleic acid or amino acid sequences and frequency determination at some or all positions of interest.

As used herein, the term “read sequence” is intended to mean the nucleotide or base sequence information of a nucleic acid that has been generated by any sequencing method. A read therefore corresponds to the sequence information obtained from one strand of a nucleic acid fragment. For example, a DNA fragment where sequence has been generated from one strand in a single reaction will result in a single read. However, multiple reads for the same DNA strand can be generated where multiple copies of that DNA fragment exist in a sequencing project or where the strand has been sequenced multiple times. A read therefore corresponds to the purine or pyrimidine base calls or sequence determinations of a particular sequencing reaction.

As used herein, the term “automated” or “automated method” is intended to mean a self-controlled operation of an apparatus, method or system by mechanical or electrical devices, or both, that can substitute for human intervention, including cognitive decision methods. Minor human interventions which do not substantially affect the primary functions of the method are included within the definition of the term. Such minor interventions can include, for example, input and export of data, including beginning and ending data. Generally, a method is automated through the control of a computer, which is a programmable electronic device that can store and retrieve data. An algorithm refers a series of procedural instructions that define the automated steps of a method. In a computerized method, the algorithm defines a list of coded instructions implemented by the computer.

Because nucleic acids encode biological information in a double-stranded, complementary form or in single-stranded forms corresponding to either a sense or a complementary anti-sense strand, those skilled in the art will understand that references herein to a nucleic acid or nucleic acid sequence describes either or both strands of a nucleic acid molecule. Therefore, two sequences can be overlapping and for that reason be complementary, for example, with respect to sense strands, anti-sense strands, complementary strands, or both as it is well known to those skilled in the art that knowledge of a single strand of nucleic acid sequence necessarily provides the complementary strand. Algorithms and automated processes are similarly well known in the art that can, for example, search, align, assemble, cluster, compare and manipulate either or both the sense and complementary strand of nucleic acid sequences. Such algorithms and automated processes similarly, for example, search, align, assemble, cluster, compare and manipulate amino acid sequences in like manner. Thus, reference to a nucleic acid or amino acid reference sequence includes a description of both its sense and complementary sequence and its translated amino acid sequence.

The invention provides an automated method for determining similarity between each of one or more template sequences and a corresponding reference sequence using computer implemented steps. The steps include (a) verifying that one or more first read sequences corresponding to one or more first portions of each of the one or more template sequences is substantially the same as a corresponding reference sequence, wherein the first read sequences are obtained by sequencing using a defined primer, and (b) confirming that one or more second read sequences corresponding to second portions of one or more verified template sequences is substantially the same as the corresponding reference sequence, wherein the second read sequences are obtained using reference sequence primers. This method can be referred to as “confirmation sequencing.”

The invention also provides modules containing instructions that can be implemented by a computer for determining the similarity between one or more template sequences and a reference sequence that corresponds to each sequence. The steps of the methods, and corresponding computer implemented instructions, advantageously combine computational search, alignment and clustering processes to overcome prohibitively slow semi-manual processes that are labor intensive or brute-force computational approaches. For example, the automated methods of the invention are substantially faster than conventional methods for determining the similarity between template and reference sequences because the automated methods can process and compare at least 4 times the number of template sequences to a corresponding reference sequence in a given time period in comparison to conventional methods.

The automated method for determining similarity between each of one or more template sequences and a corresponding reference sequence can be used to determine similarity of one or more, two or more, three or more, ten or more, fifty or more, one hundred or more, five hundred or more, one thousand or more, two thousand or more, or several thousand or more template sequences, each with a corresponding reference sequence.

The system for automated determination of the similarity between one or more template sequences and a corresponding reference sequence can process at least 36,500 reads per day, including 50 or more, 500 or more, 5000 or more, 20,000 or more, 40,000 or more, 50,000 or more and 60,000 or more reads per day. Generally, one operator of the system can perform confirmation sequencing on 12,000 or more full length subject sequences per year. The system can provide a maximum burst processing capacity of at least 78,000 reads per day, including least 5000 reads per day, at least 10,000 reads per day, at least 50,000 reads per day, or even at least 90,000 reads per day or at least 100,000 reads per day. The burst capacity can be increased by the use of additional computers or other resources. For example, The system for automated determination of the similarity between one or more template sequences and a corresponding reference sequence is scalable, with no single component causing a bottleneck that limits increased capacity. Therefore, the system capacity can be expanded by the use of additional computers.

The confirmation sequencing method of the invention can involve verifying that first read sequences corresponding to one or more first portions of each of the one or more template sequences are substantially the same as a corresponding reference sequence. A verified template read sequence is one that has been determined to be substantially the same as a corresponding reference sequence.

A template read sequence can be pre-existing or can be can determined as step of the computer implemented methods of the invention. A pre-existing or newly generated read sequence can be contained in a computer file, which can be in a variety of file formats, and can be stored using short-term or long-term storage methods, including a variety of database formats. Determining a read sequence can be implemented by computer programs such as PHRED and PHRED_qual.

PHRED is a publicly available computer program that reads DNA sequencer trace data, calls bases, assigns quality values to the bases, and writes the base calls and quality values to output files (see, for example, Ewing and Green, Genome Research 8:186-194 (1998). PHRED can read trace data from SCF, ABI model 373 and 377 DNA sequencer chromatogram, and MegaBACE ESD chromatograms files, automatically detecting the file format, and whether the chromat file was compressed by gzip or UNIX compress. After calling bases, PHRED writes the sequences to files in either FASTA format, the format suitable for XBAP, PHD format, or the SCF format. Those skilled in the art will be able to select a nucleotide sequence characterization program compatible with the output of a particular sequencing machine, and will be able to adapt an output of a sequencing machine for analysis with a variety of base-calling programs.

To verify similarity of a template read sequence with a corresponding reference sequence, the two sequences can be aligned. Generally, to determine whether any two or more nucleotide or amino acid sequences are similar, a sequence alignment can be generated using a variety of methods. An alignment of nucleic acid or amino acid sequences is a representation of two or more sequences sharing matches, mismatches or gaps at each nucleotide or amino acid position when placed in proper relative position or orientation. The degree to which positions match or correctly align is a measure of their sequence similarity. Sequences that completely match, without mismatches or gaps, are considered to be the same. In contrast, sequences that do not align, or exhibit a frequency of matching positions expected to occur by chance, are considered to be different. Sequences that align with match frequencies greater than chance are considered significant and fall within the meaning of the term as used herein. Therefore, the term “substantially” as used herein with reference to the degree of nucleic acid or amino acid sequence alignment is intended to mean that the compared sequences are the same, or are deemed to be the same, given for example, the sequencing error rate inherent in input data, the algorithm used for comparison and the search and alignment parameters employed in a particular run analysis. Given a particular computational background and sequencing data source, those skilled in the art will know, or can determine, a range or boundary of nucleotide or amino acid match that is acceptable for deeming two sequences to be substantially the same. Methods for aligning two or more nucleic acid or amino acid sequences are well known in the art. Such methods include, for example, local sequence alignment, pairwise alignment and multiple alignment. Similar alignment algorithms and written instructions for their automated implementation are well known to those skilled in the art. Such algorithms and instructions include, for example, dynamic programming, heuristic algorithms, linear space, hidden Markov models (HMM), Barton-Sternberg algorithm, profile HMMs, Feng-Doolittle progressive alignment, multidimensional dynamic programming, Smith-Waterman algorithm, Neddle and Wunsch algorithm, BLAST, FASTA, d2_cluster, Phrap, and CLUSTAL. Any of these methods, as well as others well known to those skilled in the art can be used in the automated methods of the invention.

Because a template read sequence can contain the nucleotide bases that correspond to a portion of the template sequence, the template read sequence can align with a portion of the reference sequence. Thus, the corresponding reference used for a particular alignment can be a portion of a given reference sequence or the full length reference sequence, depending on length of the template read sequence. Based on the number or percentage of template sequence nucleotide bases that are the same as or different from a corresponding reference sequence nucleotide base, a selected program can calculate a value used for assessing whether the template is substantially the same as a reference. Generally, a template sequence, such as a template read sequence, a consensus template read sequence or an assembled template sequence, is substantially the same as a reference sequence when 95% of template sequence nucleotide bases are the same as the corresponding reference sequence nucleotide bases, and can include when 98%, 99% or even 100% of template sequence nucleotide bases are the same as corresponding reference sequence nucleotide bases in the region compared.

A script or program also can be used to assign a quality score to an individual template read sequence, and, if desired, to reject a template read sequence of insufficient quality. As such, template first reads that meet a quality threshold can be subjected to verifying that first read sequences corresponding to one or more first portions of each of the one or more template sequences are substantially the same as a corresponding reference sequence. A quality value generally reflects the number of errors made by the basecaller. Such errors are often attributable to misinterpretation of peaks in a region of the trace and can be estimated by assigning values to various parameters of a chromatogram and basecalled sequence. For example, the PHRED program determines quality values based on parameters such as peak spacing, uncalled/called ratio for the entire sequence and for a window of three peaks, and peak resolution. Basecalling and quality values can be determined using a variety of other programs well-known to those skilled in the art. Exemplary programs include TRACETUNER, DNASCAN and the like. Those skilled in the art will be able to select or write a program for basecalling and determining sequence quality values. Those skilled in the art also will be able to select a program compatible with the output of a particular sequencing machine, and will be able to adapt an output of a sequencing machine for analysis with a variety of nucleotide sequence characterization programs.

A first read sequence of one or more template nucleic acids is obtained by sequencing using a defined primer, which can be a vector primer or a reference sequence primer. A vector primer is a sequencing primer that aligns with a vector sequence expected to be present in a clone containing a template sequence. A vector primer can be used when it is desired to obtain a 3′ or 5′ terminal sequence of a template sequence. When all template nucleic acids to be analyzed are contained within a common vector, the use of a vector primer is particularly convenient because sequence will be obtained whether or not a template sequence is similar to a corresponding reference sequence. A reference sequence primer is a sequencing primer that aligns with a given reference sequence. A reference sequence primer can be used when it is desired to obtain a non-terminal end portion of a template sequence. When using a reference sequence primer for obtaining a first read sequence, a primer that directs sequencing of a reference sequence at a position known to contain variable sequence, such as a region of a gene known to differ among clones due to splice variation or mutation, can be selected.

A defined primer or reference sequence primer can be used to obtain forward or reverse read sequence, and two or more defined or reference primers can both be used to obtain forward read sequence, reverse read sequence, or both forward and reverse sequence. When both forward and reverse read sequence is obtained, the read sequences can be overlapping or non-overlapping. If desired, two first reads obtained in forward and reverse directions can be aligned to determine that a first read obtained with one primer is accurate. Similarly, two second or subsequent reads can be aligned to determine that the sequence obtained with a particular primer is accurate

A template sequence can be subjected to sequencing with one or more defined primers, two or more defined primers, three or more defined primers or any number of primers as needed for a particular application of the method. The number of defined primers selected can be chosen based on the length of the template sequence and the expected variability of the template sequence in comparison to a corresponding reference sequence, with longer or more variable template sequences being verified using multiple primers. Similarly, a template sequence can be subjected to sequencing with one or more reference primers, two or more reference primers, three or more reference primers or any number of reference primers as needed for a particular application of the method. As described below, a plurality of reference primers can be used when it is desired to obtain a template read sequence to cover sequence lengths requiring more than one read sequence, including full length template sequences. A plurality of primers that are used to obtain only forward reads or only reverse reads are referred to as unidirectional primers, while a plurality of primers used to obtain both forward and reverse reads are referred to as bidirectional primers. A plurality of unidirectional primers generally are used when it is desired to obtain a single-stranded assembled template read sequence, while a plurality of bidirectional primer sets generally are used when it is desired to obtain a double-stranded assembled template read sequence, although two sets of unidirectional primers can be used in turn to obtain each strand of a double-stranded assembled template sequence, if desired.

Sequencing of a template sequence with two or more defined primers or reference primers can be performed simultaneously or consecutively. A simultaneous process has the advantage of convenient tracking of samples, while a consecutive process has the advantage of saving costs associated with sequencing. For example, a consecutive process can be used to determine a first template read sequence. The resultant first template read sequence can be subjected to verification. If the template read sequence is verified, the confirmation step can be allowed to proceed. If the template read sequence is not verified, a second defined primer can be used to obtain another first template sequence, which can be subjected to verification. Alternatively, if the template read sequence is not verified, the sequence can be compared to a different reference sequence.

In one embodiment, the confirmation sequencing method of the invention involves comparing a template read sequence to more than one reference sequence. The number of reference sequences used for comparison with a template read sequence will depend upon the application of the invention methods. For example, when the template sequence is an uncharacterized sequence, such as an open reading frame, comparison with multiple reference sequences to determine the identity of the template sequence can be performed.

The confirmation sequencing method of the invention involves confirming that second read sequences corresponding to second portions of one or more verified template sequences are substantially the same as the corresponding reference sequence. For each verified template sequence, a second read sequence contains at least a portion of template sequence that is non-overlapping with sequence obtained for a first portion of the template sequence. Therefore, a second reference primer that is non-overlapping with a defined primer used for a particular template sequence is selected.

The confirmation sequencing method of the invention also can involve identifying one or more differences between one or more template sequence read sequences and their corresponding reference sequences. Differences can be determined between individual template read sequences, consensus template read sequences, and assembled template read sequences and their corresponding reference sequences. Generation of consensus template read sequences and assembled template read sequences are described herein below. Such differences can include nucleotide base insertions, deletions, and substitutions in a template read sequence, consensus template read sequence or assembled template sequence with respect to a corresponding reference sequence.

The verification and confirmation steps of the confirmation sequencing method can be performed consecutively or simultaneously. As described above with respect to scenarios in which the confirmation sequencing methods can be used, simultaneous verification and confirmation can be useful when each template nucleic acid molecule is expected to be substantially the same as a corresponding reference sequence. In such a case, investment in materials and time to obtain read sequences from two or more portions of each template sequence can be considered as low risk, in comparison to cases in which template sequences are expected to differ from reference.

The template sequences analyzed in the methods of the invention can each have a corresponding reference sequence, such as when each template sequence is expected to be a different nucleic acid molecule. Similarly, two or more template sequences analyzed in the methods of the invention can share the same corresponding reference sequence. As described in Example II, duplicate claims analyzed using the confirmation sequencing method shared a common reference sequence. Thus, the automated method for determining similarity between each of two or more template sequences and a corresponding reference sequence can be carried out using a different reference sequence for each template sequence for each template sequence for each template sequence, a shared reference sequence for two or more templates, or for all templates analyzed, or a combination of distinct and shared reference sequnces.

The particular sequence within a reference sequence used for comparison with a template read sequence, consensus read sequence or assembled template sequence will depend upon the length of the particular read or assembled sequence determined to be of sufficient quality. The particular sequence within a reference sequence can be selected to be the same length as the particular read or assembled sequence to which it is compared. Therefore, either a portion of a reference sequence or a full-length reference sequence can be compared to a template sequence. Similarly, either a portion of an amino acid sequence encoded by a reference sequence or a full length amino acid sequence can be compared to an amino acid sequence encoded by a template sequence. The template sequences analyzed in the methods of the invention also can be expected to be all the same, and share a single corresponding reference sequence. When all template sequences are expected to be the same, such as when isolating many clones of the same gene, a consensus template sequence can be assembled to determine any portions of the sequence prone to mutation, to determine areas within the sequence that lack sequence coverage, or that lack high quality sequence coverage. When a consensus template sequence has been determined, comparison with the corresponding reference sequence can reveal that the reference sequence differs from the consensus sequence. Any such differences can be used to revise the reference sequence.

Therefore, the confirmation sequencing method of the invention also can involve determining a consensus template read sequence, including a consensus first or second template read sequence and a consensus assembled template sequence. A consensus template sequence is a template sequence having each nucleotide position determined by comparison of the nucleotides present at equivalent positions in two or more template sequences that are members of a group of sequences corresponding to the same clone. A consensus sequence is generally determined by conserving each position within a nucleotide sequence determined to be the same for each member of the group. For nucleotide positions having variability among members of the group, the most commonly occurring nucleotide at a particular position can be selected for inclusion in the consensus sequence, or the consensus sequence can contain more than one nucleotide base at a single position.

Methods for determining a consensus sequence of two or more nucleic acid sequences are well known in the art. For obtaining a consensus sequence, the method of the invention can advantageously utilize the well-known program Polyphred. Other exemplary methods for determining consensus sequences are described, for example, in G. Z. Hertz and G. D. Stormo, Proceedings of the Third International Conference on Bioinformatics and Genome Research (H. A. Lim, and C. R. Cantor, editors). World Scientific Publishing Co., Ltd., 201-216 (1995). Two or more template read sequences or assembled template sequences to be included in a consensus sequence can be grouped into an assembly group, which can be processed using a selected consensus building computer program.

The confirmation sequencing method of the invention also can involve assembling a plurality of contiguous read sequences for one or more verified template sequences to generate an assembled template sequence. Methods for assembling two or more nucleic acid sequences are well known in the art. For example, well known computer programs for aligning read sequences into contiguous single-stranded or double stranded sequences include Phrap, Arachne and Paracel. Any of these methods, as well as others well known to those skilled in the art can be used in the automated methods of the invention.

If it is desired to compare a more extensive portion of a template sequence with a reference sequence, additional template read sequence corresponding to missing or inaccurate read sequence can be obtained using the automated methods of the invention. Therefore, the method for determining an assembled template sequence can involve (i) selecting a plurality of references sequence primers; (ii) generating a template read sequence for each of the primers; (iii) assembling two or more of template read sequences to obtain an assembled template sequence. Steps (i) through (iii) can be repeated to assemble a full length template sequence, if desired.

For cases in which two or more template read sequences are not efficiently assembled into a full-length contiguous sequence, or into a desired contiguous portion of a template sequence, additional reference sequence primers can be selected to obtain read sequence over a particular portion of a template sequence. Selected primers can be used for obtaining forward or reverse template sequence, or both forward and reverse sequence, depending on whether it is desired to generate a single strand of a template sequence or both strands of a template sequence. As described below, the Primer Picking Module of the system of the invention provides an automated process for selecting reference sequence primers, and even can be used for ordering the preparation of the selected primers.

The confirmation sequencing method of the invention also can involve identifying one or more differences between an amino acid sequence encoded by one or more template read sequences, consensus template sequences and assembled template sequences, and an amino acid sequence encoded by the reference sequence.

Methods for translating a nucleotide sequence into an amino acid sequence, and for determining differences between two or more amino acid sequences are well known in the art. For example, well known computer programs useful for comparing amino acid sequences include BLASTP, FASTA, SSEARCH, and BLITZ. As described below, the Sequence Comparison Module of the system of the invention provides an automated process for determining one or more differences between an amino acid sequence encoded by one or more template read sequences, consensus template sequences or assembled template sequences and an amino acid sequence, or portion thereof, encoded by the reference sequence.

The invention provides an automated method for identifying differences between one or more verified template sequences and a corresponding reference sequence. The method includes the computer implemented steps of (a) verifying that a first read sequence corresponding to a portion of each of the one or more template sequences is substantially the same as a corresponding reference sequence, wherein the first read sequence is obtained by sequencing using a defined primer; (b) confirming that a second read sequence corresponding to a second portion of one or more verified template sequence is substantially the same as the corresponding reference sequence, wherein the second read sequence is obtained by sequencing using a reference sequence primer, and (c) identifying one or more differences between one or more verified template nucleotide sequences and the corresponding reference sequence.

The invention provides an automated method for determining similarity between one or more full length template sequences and a corresponding reference sequence. The method includes the computer implemented steps of (a) verifying that a first read sequence corresponding to a portion of each of the one or more template sequences is substantially the same as a corresponding reference sequence, wherein the first read sequence is obtained by sequencing using a defined primer; (b) confirming that a second read sequence corresponding to a second portion of each verified template sequence is substantially the same as the corresponding reference sequence, wherein the second read sequence is obtained by sequencing using a reference sequence primer; (c) assembling a plurality of contiguous read sequences to generate a full length template nucleotide sequence for each verified template sequence, the read sequences obtained using a plurality of reference sequence primers, and (d) identifying one or more differences between one or more assembled template nucleotide sequences and the corresponding reference sequence.

The invention further provides a computer readable medium comprising instructions, which when executed on a processor, implement a method having the steps: (a) verifying that one or more first read sequences corresponding to one or more first portions of each of the two or more template sequences is substantially the same as a corresponding reference sequence, wherein the first read sequences are obtained by sequencing using a defined primer, and (b) confirming that one or more second read sequences corresponding to second portions of one or more verified template sequences is substantially the same as the corresponding reference sequence, wherein the second read sequences are obtained using reference sequence primers.

A computer readable medium can be a hard disk, floppy disc, compact disc, magneto-optical disc, Random Access Memory, Read Only Memory or Flash Memory. A computer system that contains the computer readable medium used in the invention can be a single computer or multiple computers distributed in a network.

The invention provides a system for carrying out the computer implemented method for determining similarity between two or more template sequences and a corresponding reference sequence. The Confirmation Sequencing System of the invention functions to enhance the throughput and accuracy of determining similarity between a template sequence and corresponding reference sequence compared to conventional semi-automated methods that require human intervention. The system automates previously manual operations, including verification and confirmation of the similarity of a template sequence and a reference sequence. Briefly, the Confirmation Sequencing System can include a variety of modules for performing computer-implemented steps of the invention methods. Exemplary modules include a pre-processing module, an assembly processor module, an assembly reporter module, a verification module, a confirmation sequencing module, a sequence comparison module, a consensus building module, a status summary module, a primer picking module, a data delivery module, a distribution client module, and a sequence loading module.

A pre-processing module functions to place read sequences and one or more corresponding reference sequences in a common location for processing. For example, a script or program can be used to write one or more reference sequences into a location, such as a directory or file, containing run sequence files, and to export read sequence files from a database into a directory or file. Those skilled in the art can select a script or program for moving files into locations where the files can be obtained for processing.

An assembly processor module functions to assemble two or more read sequences together to generate an assembled read sequence. As such, the assembly processor module contains scripts and programs that can identify files containing read sequences to be assembled, and can align two or more overlapping read sequences to obtain a contiguous sequence, referred to as an assembled sequence. The assembly processor module can assemble template read sequences, including first or second template read sequences, consensus template read sequences and assembled template read sequences. A variety of well-known programs for assembling sequences can be included in the assembly processor module. Examples of such assembly programs include PhredPhrap, Phrap, Arachne, and Paracel. An assembly processor module can include, for example, instructions for automatic assembly of a new sequence placed in a defined location, such as a specified assembly directory. An assembly processor module can include instructions for computing assembly statistics, including, for example, average base coverage, number of Phred-20 bases, number of external mates, individual base quality, average error probability and coverage direction. An assembly processor module also can include instructions for a messaging system whereby an individual is informed, such as by e-mail, of information relevant to a particular sequence or set of sequences that has been assembled. An assembly processor module further can include a viewer that allows a user to view an assembled sequence. Instructions in the assembly processor module can extract information from a file to identify the location on the system where an assembly is to take place. Those skilled in the art can select a script or program for inclusion in an assembly processor module, and will know how to link together scripts or programs within the assembly processor module as desired using scripts appropriate for the particular platform and software used.

An assembly reporter module functions to create a report describing results of an assembly attempt. Such a report can be based on, for example, a directory path where an assembly has occurred, and can describe results of assembling read sequences. A report can describe such results with respect to read sequences, template sequences, projects, users, and the like. Those skilled in the art can select a script or program for inclusion in an assembly reporter module, and will know how to link together scripts or programs within the assembly reporter module as desired using scripts appropriate for the particular platform and software used.

A verification module functions to whether one or more template read sequences are substantially the same as their corresponding reference sequences. In one scheme, a verification module can be a program or series of scripts that takes sequence file identifying information, such as a project name and a list of directory paths where read sequence files were delivered and where the reference sequence for one or more template sequences exists, and invoke a sequence alignment and comparison program, such as cross_match, swat, or the like, to compare the template read sequences with one or more corresponding reference sequences, and determine whether one or more reference sequences are substantially the same as their corresponding references sequences. The verification module also can function to generate a report to describe similarity using such indicators as yes/no; percent identity; percent identity within a defined portion of a template sequence; percent identify over an entire read; and the like.

Exemplary processing steps that can be invoked by the verification module include: retrieving sequence files, for example, files having a particular date or project name; updating a list to record first time sequencing; copying sequence files to assign a customer naming convention and writing the files to a directory; running a base calling program on the reads, for example, using a program such as Phred; screening to identify low quality sequence; clipping or masking to remove low quality sequence; comparing template sequence read sequences with corresponding reference sequences, for example, using cross_match or swat; comparing template sequence read sequences with a second reference sequence, and generating a report listing similarity information, including a customer or internal use report, or both. An exemplary report can include a desired amount of information, from a single data element, such as a list of template sequences determined to be substantially the same as a reference sequence, or a list of template sequences determined to be different from a reference sequence; to a few data elements, such as a sequence identifying name, details of the similarity between a template sequence and corresponding reference; characterization of differences between a template and reference sequence; file names, for example, for 5′ and 3′ read sequences; many data elements, or all available data elements.

Another exemplary process carried out by the verification module of the system of the invention is as follows: (1) Retrieve sequence files for a particular date. The sequence files can be in any convenient format, such as FASTA or SCF files. (2) Update a list to record first time sequencing. The list can be further updated for second and subsequent rounds of sequencing, if relevant. (3) Copy the retrieved files, translating to customer naming convention and writing to a directory, such as a directory specific for a project name, subproject, or assembly group. (4) Run Phred on reads to obtain base calls, optionally running screening and clipping functions of Phred. (5) Run cross_match on the reads and corresponding reference sequences. (6) Determine if sequence is similar to reference and parse cross_match output. (7) Create report listing successful/unsuccessful match information, such as in a customer specified format. (8) Create a report listing more comprehensive match information for internal use. This process has been used to verify and report on the successful or unsuccessful match of each clone in a population to a corresponding reference sequence. Those skilled in the art can select a script or program for inclusion in the verification module, and will know how to link together scripts or programs within the verification module as desired using scripts appropriate for the particular platform and software used.

A confirmation sequencing module functions to determine if one or more second portions of one or more template sequences, including consensus template sequences and assembled template sequences, are substantially the same as their more corresponding reference sequences. This module can, for example, invoke processing by taking sequence file information, such as a project and a list of directory paths, to a location, such as a directory, table or file, where confirmation sequencing testing will be performed. A confirmation sequencing module can be invoked subsequent to the verification module, or independently of the verification module. As such, exemplary processing steps that can be invoked by the confirmation sequencing module include: retrieving sequence files, for example, files having a particular date or project name; updating a list to record first time sequencing; copying sequence files to assign a customer naming convention; writing the files to a file or directory; running a base calling program on the reads, for example, using a program such as Phred; screening to identify low quality sequence; clipping or masking to remove low quality sequence; comparing template sequence read sequences with corresponding reference sequences, for example, using cross_match or swat; comparing template sequence read sequences with a second reference sequence; selecting quality reads for inclusion in an assembly; selecting reads of a particular length for inclusion in an assembly; assembling a plurality of read sequences to generate a longer portion of a template nucleotide sequence, including a full length template nucleotide sequence; determining if an assembled template nucleotide sequence is complete in comparison to a corresponding reference sequence; and generate a report listing similarity information, including a customer or internal use report, or both. An exemplary report can include a sequence identifying name, an indication of the similarity between a template sequence and corresponding reference, a location within a sequence of a difference between a template sequence and reference sequence, a specific difference between a template sequence and reference sequence, such as a base change; file names, for example, for 5′ and 3′ read sequences. Those skilled in the art can select a script or program for inclusion in the confirmation sequencing module, and will know how to link together scripts or programs within the module as desired using scripts appropriate for the particular platform and software used.

A sequence comparison module functions to identify differences between template nucleotide or amino acid sequences and corresponding reference nucleotide or amino acid sequences. As such, the sequence comparison module can contain scripts and programs that can align two or more template sequences with a corresponding reference sequence and identify any differences between the template and reference sequences, such as mismatches, insertions and deletions. A sequence comparison module also can contain scripts and programs that translate a selected nucleotide sequence into an amino acid sequence and compare a template amino acid sequence with a reference amino acid sequence. A sequence comparison module further can contain scripts and programs that can characterize a particular amino acid change in a template amino acid sequence relative to a reference amino acid sequence, for example, by determining that a change corresponds to a conservative substitution or non-conservative substitution. A sequence comparison module can be contained within a verification module and a confirmation sequencing module, or can be used independently of these modules, depending on the particular application of the method. For example, a sequence comparison module can be contained within a verification or confirmation sequencing module when template sequences are to be compared with reference sequences as part of a verification or confirmation step of the methods of the invention. Alternatively, a sequence comparison module can be used to compare a template amino acid sequence with a reference amino acid sequence subsequent to running of a confirmation sequencing module. Those skilled in the art can select a script or program for inclusion in the sequence comparison module, and will know how to link together scripts or programs within the verification module as desired using scripts appropriate for the particular platform and software used.

A consensus building module functions to determine a consensus sequence corresponding to two or more read sequences, including single reads and assembled read sequences. As such, a consensus building module contains written instructions, such as scripts and programs, that can identify sequence files to be aligned for determining a consensus sequence, can align the selected read sequences and can determine a consensus sequence. A variety of well-known algorithms can be used to determine a consensus sequence from two or more template sequences that overlap. Such algorithms include consensus calculations using base frequencies, using weighted base frequencies, and using confidence values. A suitable algorithm can be selected by those skilled in the art. Similarly, those skilled in the art can select a script or program for inclusion in the consensus building module, and will know how to link together scripts or programs within the module as desired using scripts appropriate for the particular platform and software used.

A primer picking module functions to select reference sequences or complementary reference sequences for use as primers for sequencing a template sequence to obtain a portion of the template sequence that is absent or of insufficient quality. As such, the primer picking module can contain scripts and programs that can determine a portion of template sequence missing from a template read sequence or an assembled template sequence, and can select primer sequences from the corresponding reference sequence. Selected primers are those that are predicted to result in a sequencing run that generates one or more template read sequences that encompass one or more portions of missing or insufficient quality template sequence. In an exemplary primer picking application, a user can submit a file containing the names and sequences of the relevant reference sequences, as well as such values as type of coverage required, such as single or double-stranded sequencing, and the desired interval between selected primers. The application can then calculate the size of each reference and select an appropriate number of primers to meet the specific coverage requirements. The application also can generate names for the primers that incorporate directional information. Such names can be used in the primer picking module to distinguish between forward and reverse reads. Those skilled in the art can select a script or program for inclusion in the primer picking module, and will know how to link together scripts or programs within the module as desired using scripts appropriate for the particular platform and software used.

A status summary module functions to report information on the progress of method steps performed in any module of the system. As such, the status summary module can contain written instructions, such as scripts, that report the status of files in particular directories. For example, a status summary module can include instructions for reporting information on assembly groups that have been processed, for consensus sequences generated, for sequences aligned, for differences between sequences identified, and the like. Those skilled in the art can select a script or program for inclusion in the status summary module, and will know how to link together scripts or programs within the module as desired using scripts appropriate for the particular platform and software used.

A data delivery module functions to delivery files to a particular location. As such, the data delivery module can contain written instructions, such as scripts, that direct transfer or relocation of a file to another location, for example, from one directory to another. Those skilled in the art can select a script or program for inclusion in the data delivery module, and will know how to link together scripts or programs within the module as desired using scripts appropriate for the particular platform and software used.

A sequence loading module functions to load, update or both load and update sequences, such as reference sequences and template sequences to be processed. As such, the sequence loading module contains written instructions, such as scripts, that direct transfer of one or more files into a database. The sequence loading module can provide the advantages of minimized random disk accesses, minimized disk I/O, minimized CPU load, optimized clustering and optimized page filing, in comparison to using a standard insert operation of a database. Using the sequence loading module, multiple sequence files or multiple sequences contained in a single file can be loaded into a database. A specific method for sequence loading can be selected from well known loading methods based on desired performance criteria, such as elapsed time, bandwidth, memory consumption, and the database or platform employed.

An exemplary sequence loading module is a bulk loading module, which can be used for initial bulk loading and incremental bulk loading. Any bulk loading algorithm can be included in the sequence loading module. For example, algorithms that apply a certain partition to multidimensional input data and load those partitions into the database, and algorithms that apply a total sort order to the input data and load the pre-sorted data into the database can be used. The sequence loading module can be used to load all reads, or a subset of reads into a particular directory or database. The sequencing loading module can include a mechanism for selecting sequences to be loaded, such as activation threshold criteria, which is an expression among the attributes of a read, such as sequence name, clipped length, estimated error of clipped sequence, estimated error of unclipped sequence, count of high quality bases, quality average, quality maximum, quality minimum, count of very high quality bases, and the like. For example, sequences can be tested against the activation criteria, and ones that match the criteria can be made active, while non-matching sequences can be made inactive, with active sequences being loaded into the database. The sequence loading module also can include a filter for selecting sequences to be loaded into a database.

The sequence loading module can load sequences in a variety of file types, including for example a single tab-delimited file or multiple individual files, into a database. Sequences can be in a variety of formats, including, for example, FASTA, PHD or other convenient format, and can be annotated, if desired. Information related to a sequence, including, for example, an identification number, sequence name, project, type of file, owner, user, viewer, data source, and date, also can be loaded into a database. Those skilled in the art can select a script or program for inclusion in the sequence loading module, and will know how to link together scripts or programs within the module as desired using scripts appropriate for the particular platform and software used.

To perform a computer implemented method of the invention, one or more distribution clients can be employed. A distribution client can be used, for example, to invoke specific sequence processing; to select sequences for processing based on selection criteria; to invoke user scripts, and to deliver output information to a selected location. Therefore, the system can include a distribution processing module that provides instructions for defining a Distribution Client. The Distribution Client can specify selection of a type of sequence, a manner of sequence analysis for the selected sequence and a delivery location of sequence analysis output for the selected sequence. A Distribution Client can be a default, predetermined or user-selected Distribution Client that specifies a set of sequence analysis and distribution actions within the superset.

A Distribution Client can include: (a) a Distribution Selector having a selection algorithm; (b) a Command Set invoking one or more sequence analysis procedures; and (c) a Distribution File Specification having user controlled delivery location of a sequence analysis output.

A Distribution Selector is a representation of a boolean expression involving the attributes of the sequence and its core statistics. The purpose of the Distribution Selector is to select some sequences for processing by a Distribution Client. Sequences flowing through the system can be tested against each Distribution Selector, and those that result in a boolean true result can get further processing within those Distribution Clients.

The selection algorithm can select a sequence type specified by one or more sequence attributes for further processing by a Distribution Client. A variety of sequence attributes, such as Generationcount, Identifier, LimsSource, Location, MachineName, MachineType, ProcessDate, RunDate RunFolderName, Status, AssemblyGroup, ChargeNumber, DataFormat, DataLocation, Direction, ExperimentId, Group Identifier, LibraryId, LibraryName, Name, PipeSeqName, PlateName, Project, QualityLocation, SeqReactionChemistry Status, Strandedness, SubmissionDate, SubmissionId Subproject, TemplateCloneType, TemplateId, TemplateName UserLabel, ClassifiedAs, ClippedLength, EstErrorClipped, EstErrorUnclipped, HQBases, ProcessDate, QualityAverage Quality Maximum, QualityMinimum, Status, UnclipLength, VectorBases, VHQBases)or any combination thereof, can be specified. The selection algorithm can be, for example, a boolean logic algorithm.

A Distribution File Specification can be used to allow an end-user to control the destination directory and name of each output file. This control can be expressed in the Distribution File Specification, and using it the end user can direct sequence files into different directories based on attributes of the particular sequence being distributed. For example, a user can specify literal text or the current date as part of the destination directory path.

A Distribution Client also can have an owner and a list of users to receive notification of completed processing. As an example, the owner can be a username, and the notification list can be a list of usernames. A Distribution Client also can include instructions for storing distribution statistics characterizing the sequence analysis outputs of a specified Distribution Client.

The Distribution Processing Module can provide a means to edit a Distribution Client. The implementation can, for example, edit in place, or logically delete and redefine the Distribution Client.

The Distribution Processing Module can provide a means to name or otherwise uniquely identify a Distribution Client. Such a name or identifier can be used in Reports or in services provided by the Software Integration API.

The system of the invention can include a workload management program application, such as LSF. LSF is a program that runs batch or interactive jobs, selecting execution hosts based on current load conditions and the resource requirements of the application. Interactive jobs start running as soon as a command is entered. Batch jobs are kept in queues until the appropriate resources are available. Hosts are divided in clusters. LSF provides features to make transparent access to resources in separate clusters, such as PLUS or WGS (interactive hosts used for submission) and SHIFT (batch execution clusters).

An exemplary process involving the use of a Distribution Client carried out by the system of the invention is as follows: (1) the Distribution Client processes sequence files, including sequencing machine generated filed, and delivers them to a target directory (steps can be carried out by the Pre-Processing Module). (2) A program launches a user command script into Load Sharing Facility (LSF) under the user's identity. LSF can dynamically calculate the priority of each user/group and commence processing accordingly (steps can be carried out by the Sequence Loading Module). (3) The user command script invokes Phred and Phrap. At this point, an assembly can be viewed with Consed (steps can be carried out by the Assembly Processor Module, Verification Module and/or Confirmation Sequencing Module). (4) The script invokes Cross_match to perform verification and confirmation steps, parsing the cross_match output file and extracting the relevant information (steps can be carried out by the Verification Module and Confirmation Sequencing Module). (5) The script sends email containing results of the verification and confirmation steps to the user (steps can be carried out by the Data Delivery Module).

Instructions contained in a module of the system of the invention can be in the form of a computer program, such as a shell script, Perl script Java program, or any other program compatible with a selected platform, such as SGI Irix, Sun Solaris/Sun OS, Digital Unix, Hewlett-Packard's HP-UX, and Windows NT. Instructions, can, for example run in parallel or in series, for example, on shared-memory multi-CPU computer systems. Those skilled in the art can select a script or program for linking together scripts or programs between two or more different modules of the system.

The Confirmation Sequencing system of the invention can present results to end users through a graphics user interface (GUI), if desired. Results generated in one module also can be used by another module. Results obtained by running a module of a system of the invention also can be stored for short-term or long-term. As such, the system of the invention for determining similarity between each of two or more template sequences and a corresponding reference sequence can include one or more databases, which can be distributed databases. For large data sets, it can be advantageous to interface one or more databases to archival storage.

It is understood that those skilled in the art will know, or can determine, that the system architecture and configuration as well as the functions of the modules and components described above can be simulated or performed by other structures and logic well known to those skilled in the art. Therefore, using the teachings and guidance described herein, functional substitutions and minor modifications of the structures and components described below for the Confirmation Sequencing System can be made by those skilled in the art and still be encompassed by the Confirmation Sequencing System of the invention. For example, the Confirmation Sequencing System is described with reference to determining whether a template sequence has the same nucleotide sequence as a reference sequence. However, those skilled in the art will understand that the system is applicable to all types of sequence comparisons in general as well. For example, the Confirmation Sequencing System can be implemented in the field of comparative genomics because the relatedness of compared nucleic acids sequences is a central issue to this type of discovery science.

It is understood that modifications which do not substantially affect the activity of the various embodiments of this invention are also included within the definition of the invention provided herein. Accordingly, the following examples are intended to illustrate but not limit the present invention.

### EXAMPLE I

### Use of Confirmation Sequencing to Identify Discrepencies in a Reference Sequence

To determine the accuracy of cloned DNA sequences and a corresponding reference sequence, the process of confirmation sequencing was performed.

Sequence data from the PathoGenome database was used to generate primers for isolating about 300 clones corresponding to target genes. To verify that each clone was correctly cloned into an expression vector, 5′ and 3′ sequence was obtained for each clone and compared to vector reference sequences. About 51% of completed clones were determined to be different from corresponding reference sequence. Using the methods and systems of the invention, confirmation sequencing of about 300 clones can be accomplished in about 30 to 45 days, or less, whereas conventional semi-automated methods for sequence verification typically require about six months for processing of 300 clones.

In summary, confirmation sequencing was a fast and efficient method for determining that about half of a customer's 300 cloned sequences were different from a reference sequence. These results demonstrate the usefulness of confirmation sequencing for quality control of cloned sequences.

### EXAMPLE II

### Use of Confirmation Sequencing to Determine the Correctness of Duplicate Clones

This example shows the application of confirmation sequencing to quality control of cloning processes.

Duplicate clones believed to be the same as each of 192 corresponding reference sequences were analyzed using confirmation sequencing. The verification step was used to determine that 33% of clones were different from corresponding reference sequences.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention.

What is claimed is:

1. An automated method for determining similarity between each of one or more template sequences and a corresponding reference sequence, comprising the computer implemented steps of:
(a) verifying that one or more first read sequences corresponding to one or more first portions of each of said one or more template sequences is substantially the same as a corresponding reference sequence, wherein said first read sequences are obtained by sequencing using a defined primer, and
(b) confirming that one or more second read sequences corresponding to second portions of one or more verified template sequences is substantially the same as said corresponding reference sequence, wherein said second read sequences are obtained using reference sequence primers.

2. The method of claim 1, wherein one or more of said defined primers is a vector sequence primer.

3. The method of claim 1, wherein one or more of said defined primers is a reference sequence primer.

4. The method of claim 1, wherein one or more of said defined primers is a 5′ terminal primer.

5. The method of claim 1, wherein one or more of said defined primers is a 3′ terminal primer.

6. The method of claim 1, wherein said verifying is performed using two or more first read sequences.

7. The method of claim 6, wherein said defined primers include a 5′ terminal primer and a 3′ terminal primer.

8. The method of claim 1, wherein said one or more reference sequence primers is a forward primer.

9. The method of claim 1, wherein said one or more reference sequence primers is a reverse primer.

10. The method of claim 1, wherein said one or more reference sequence primers is an internal reference primer.

11. The method of claim 1, wherein similarity between each of thirty or more template sequences and a corresponding reference sequence is determined.

12. The method of claim 1, wherein similarity between each of one hundred or more template sequences and a corresponding reference sequence is determined.

13. The method of claim 1, wherein similarity between each of one thousand or more template sequences and a corresponding reference sequence is determined.

14. The method of claim 1, wherein step (a) further comprises verifying that said one or more first read sequences are high quality read sequences.

15. The method of claim 14, wherein a Phred computer program is used to identify one or more high quality read sequences.

16. The method of claim 1, further comprising identifying one or more differences between one or more template sequence read sequences and said corresponding reference sequences.

17. The method of claim 1, further comprising determining a consensus first or second read sequence.

18. The method of claim 17, further comprising identifying one or more differences between one or more template consensus sequences and said corresponding reference sequence.

19. The method of claim 16 or 18, wherein said differences are selected from the group consisting of an insertion, a deletion, and a substitution.

20. The method of claim 16 or 18, wherein said differences are determined using a computer program selected from the group consisting of cross_match, SPS cross_match and swat.

21. The method of claim 16 or 18 further comprising, identifying one or more differences between an amino acid sequence encoded by one or more template read sequences or template consensus sequences and an amino acid sequence encoded by said reference sequence.

22. The method of claim 1, further comprising assembling a plurality of contiguous read sequences for one or more verified template sequences to generate an assembled template sequence, said read sequences obtained using a plurality of reference sequence primers.

23. The method of claim 22, further comprising,
(i) selecting a plurality of references sequence primers;
(ii) generating a template read sequence for each of said primers, and
(iii) assembling two or more of said template read sequences.

24. The method of claim 23, further comprising repeating steps (i) to (iii) to assemble a full length template sequence.

25. The method of claim 23, wherein said plurality of reference sequence primers are unidirectional primers.

26. The method of claim 23, wherein said plurality of reference sequence primers are bidirectional primers.

27. The method of claim 23, wherein said plurality of read sequences are forward read sequences.

28. The method of claim 23, wherein said plurality of read sequence are reverse read sequences.

29. The method of claim 23, wherein a single-stranded assembled template sequence is generated.

30. The method of claim 23, wherein a double-stranded assembled template sequence is generated.

31. The method of claim 23, wherein said plurality of read sequences are assembled using a computer program selected from the group consisting of Phrap, Arachne, and Paracel.

32. The method of claim 23, further comprising determining a consensus assembled template sequence by comparing two or more assembled template nucleotide sequences for a selected template sequence.

33. The method of claim 23, further comprising identifying one or more differences between one or more assembled template sequences and said corresponding reference sequences.

34. The method of claim 33, wherein said differences are determined using a computer program selected from the group consisting of cross_match, SPS cross_match and swat.

35. The method of claim 23, further comprising, identifying one or more differences between an amino acid sequence encoded by one or more assembled template sequences and an amino acid sequence encoded by said reference sequence.

36. An automated method for determining differences between one or more template sequences and a corresponding reference sequence, comprising the computer implemented steps of:
(a) verifying that a first read sequence corresponding to a portion of each of said one or more template sequences is substantially the same as a corresponding reference sequence, wherein said first read sequence is obtained by sequencing using a defined primer;
(b) confirming that a second read sequence corresponding to a second portion of one or more verified template sequence is substantially the same as said corresponding reference sequence, wherein said second read sequence is obtained by sequencing using a reference sequence primer, and
(c) identifying one or more differences between one or more verified template nucleotide sequences and said corresponding reference sequence.

37. The method of claim 36, wherein one or more of said defined primers is a vector sequence primer.

38. The method of claim 36, wherein one or more of said defined primers is a reference sequence primer.

39. The method of claim 36, wherein said verifying is performed using two or more first read sequences.

40. The method of claim 36, wherein step (a) further comprises verifying that said one or more first read sequences are high quality read sequences.

41. The method of claim 36, wherein a Phred computer program is used to identify one or more high quality read sequences.

42. The method of claim 36, further comprising determining a consensus first or second read sequence.

43. The method of claim 36, wherein said differences are determined using a computer program selected from the group consisting of cross_match, SPS cross_match and swat.

44. The method of claim 36 further comprising, identifying one or more differences between an amino acid sequence encoded by one or more template read sequences or template consensus sequences and an amino acid sequence encoded by said reference sequence.

45. An automated method for determining similarity between one or more full length template sequences and a corresponding reference sequence, comprising the computer implemented steps of:
(a) verifying that a first read sequence corresponding to a portion of each of said one or more template sequences is substantially the same as a corresponding reference sequence, wherein said first read sequence is obtained by sequencing using a defined primer;
(b) confirming that a second read sequence corresponding to a second portion of each verified template sequence is substantially the same as said corresponding reference sequence, wherein said second read sequence is obtained by sequencing using a reference sequence primer, and
(c) assembling a plurality of contiguous read sequences to generate a full length template nucleotide sequence for each verified template sequence, said read sequences obtained using a plurality of reference sequence primers, and
(d) identifying one or more differences between one or more assembled template nucleotide sequences and said corresponding reference sequence.

46. The method of claim 45, wherein one or more of said defined primers is a vector sequence primer.

47. The method of claim 45, wherein one or more of said defined primers is a reference sequence primer.

48. The method of claim 45, wherein said verifying is performed using two or more first read sequences.

49. The method of claim 45, wherein step (a) further comprises verifying that said one or more first read sequences are high quality read sequences.

50. The method of claim 49, wherein a Phred computer program is used to identify one or more high quality read sequences.

51. The method of claim 45, further comprising determining a consensus first or second read sequence.

52. The method of claim 45, wherein said differences are determined using a computer program selected from the group consisting of cross_match, SPS cross_match and swat.

53. The method of claim 45, further comprising, identifying one or more differences between an amino acid sequence encoded by one or more template read sequences or template consensus sequences and an amino acid sequence encoded by said reference sequence.

54. The method of claim 45, further comprising,
(i) selecting a plurality of references sequence primers;
(ii) generating a template read sequence for each of said primers, and
(iii) assembling two or more of said template read sequences.
(iv) repeating steps (i) to (iii) to assemble a full length template sequence.

55. The method of claim 54, wherein said plurality of reference sequence primers are unidirectional primers.

56. The method of claim 54, wherein said plurality of reference sequence primers are bidirectional primers.

57. The method of claim 54, wherein a single-stranded assembled template sequence is generated.

58. The method of claim 54, wherein a double-stranded assembled template sequence is generated.

59. The method of claim 54, wherein said plurality of read sequences are assembled using a computer program selected from the group consisting of Phrap, Arachne, and Paracel.

60. The method of claim 54, further comprising determining a consensus assembled template sequence by comparing two or more assembled template nucleotide sequences for a selected template sequence.

61. The method of claim 45, wherein said differences are determined using a computer program selected from the group consisting of cross_match, SPS cross_match and swat.

62. The method of claim 45, further comprising, identifying one or more differences between an amino acid sequence encoded by one or more assembled template sequences and an amino acid sequence encoded by said reference sequence.

63. A computer readable medium comprising instructions, which when executed on a processor, implement a method comprising the computer implemented steps:
(a) verifying that one or more first read sequences corresponding to one or more first portions of each of said two or more template sequences is substantially the same as a corresponding reference sequence, wherein said first read sequences are obtained by sequencing using a defined primer, and
(b) confirming that one or more second read sequences corresponding to second portions of one or more verified template sequences is substantially the same as said corresponding reference sequence, wherein said second read sequences are obtained using reference sequence primers.

64. The method of claim 63, wherein step (a) further comprises verifying that said one or more first read sequences are high quality read sequences.

65. The method of claim 63, further comprising identifying one or more differences between one or more template sequence read sequences and said corresponding reference sequences.

66. The method of claim 63, further comprising determining a consensus first or second read sequence.

67. The method of claim 63, further comprising identifying one or more differences between one or more template consensus sequences and said corresponding reference sequence.

68. The method of claim 63 further comprising, identifying one or more differences between an amino acid sequence encoded by one or more template read sequences or template consensus sequences and an amino acid sequence encoded by said reference sequence.

69. The method of claim 63, further comprising assembling a plurality of contiguous read sequences for one or more verified template sequences to generate an assembled template sequence, said read sequences obtained using a plurality of reference sequence primers.

70. The method of claim 63, further comprising,
(i) selecting a plurality of references sequence primers;
(ii) generating a template read sequence for each of said primers, and
(iii) assembling two or more of said template read sequences.

71. The method of claim 63, further comprising repeating steps (i) to (iii) to assemble a full length template sequence.

72. The method of claim 63, further comprising determining a consensus assembled template sequence by comparing two or more assembled template nucleotide sequences for a selected template sequence.

73. The method of claim 63, further comprising identifying one or more differences between one or more assembled template sequences and said corresponding reference sequences.

74. The method of claim 63, further comprising, identifying one or more differences between an amino acid sequence encoded by one or more assembled template sequences and an amino acid sequence encoded by said reference sequence.

75. An automated system for determining similarity between each of two or more template sequences and a corresponding reference sequence, comprising the computer implemented steps of:
(a) a verification module to verify that one or more first read sequences corresponding to one or more first portions of each of said two or more template sequences is substantially the same as a corresponding reference sequence, wherein said first read sequences are obtained by sequencing using a defined primer, and
(b) a confirmation sequencing module to confirm that one or more second read sequences corresponding to second portions of one or more verified template sequences is substantially the same as said corresponding reference sequence, wherein said second read sequences are obtained using reference sequence primers.

76. The system of claim 75, wherein one or more of said defined primers is a vector sequence primer.

77. The system of claim 75, wherein one or more of said defined primers is a reference sequence primer.

78. The system of claim 75, further comprising a Phred computer program for identifying one or more high quality read sequences.

79. The system of claim 75, further comprising a sequence comparison module to identify one or more differences between one or more template sequence read sequences and said corresponding reference sequences.

80. The system of claim 75, further comprising a consensus builder module.

81. The system method of claim 75, further comprising a computer program selected from the group consisting of cross_match, SPS cross_match and swat.

82. The system of claim 75, wherein said differences are differences between amino acid sequences encoded by one or more template read sequences or template consensus sequences and a reference amino acid sequence.

83. The system of claim 75, further comprising an assembly processing module to assemble a plurality of contiguous read sequences for one or more verified template sequences to generate an assembled template sequence, said read sequences obtained using a plurality of reference sequence primers.

84. The system of claim 75, further comprising a primer picking module.

85. The system of claim 75, wherein a single-stranded assembled template sequence is generated.

86. The system of claim 75, wherein a double-stranded assembled template sequence is generated.

87. The system of claim 75, further comprising a computer program selected from the group consisting of Phrap, Arachne, and Paracel.

88. The system method of claim 75, further comprising a concensus building module.

89. The system method of claim 75, further comprising a pre-processing module.

90. The system method of claim 75, further comprising an assembly processor module.

91. The system method of claim 75, further comprising an assembly reporter module.

92. The system method of claim 75, further comprising a status summary module.

93. The system method of claim 75, further comprising a data delivery module.

94. The system method of claim 75, further comprising a sequence loading module.

95. The system method of claim 75, further comprising a distribution client module.
